(19) United States
(12) Patent Application Publication
(10) Pub. No.: US 2012/0209310 A1
(43) Pub. Date: Aug. 16, 2012

(54) VASO-OCCLUSIVE DEVICE DELIVERY SYSTEM

(75) Inventors: Hancun CHEN, San Ramon, CA (US); Richard Murphy, Sunnyvale, CA (US)

(73) Assignees: Stryker NV Operations Limited, Dublin (IE); Stryker Corporation, Kalamazoo, MI (US)

(21) Appl. No.: 13/369,039

(22) Filed: Feb. 8, 2012

Related U.S. Application Data
(60) Provisional application No. 61/441,541, filed on Feb. 10, 2011.

(51) Int. Cl. A61M 29/00

(52) U.S. Cl. 606/195

(57) ABSTRACT

A vaso-occlusive device delivery system includes a delivery wire assembly having a core wire disposed in a delivery wire lumen, and a vaso-occlusive device having a tapered proximal end and defining a vaso-occlusive device lumen, wherein a tether is disposed in the lumen and secured to the core wire. A link may be attached to each of the core wire, the proximal end of the vaso-occlusive device, and the tether. Alternatively, the system may include a transition member attached to the vaso-occlusive device and secured to the core wire.

### RELATED APPLICATION DATA

The present application claims the benefit under 35 U.S.C. §119 to U.S. Provisional Application No. 61/441,541, filed Feb. 10, 2011, the contents of which are incorporated herein by reference as though set forth in full.

### FIELD

The field of the disclosed inventions generally relates to systems and delivery devices for implanting vaso-occlusive devices for establishing an embolus or vascular occlusion in a vessel of a human or veterinary patient. More particularly, the disclosed inventions relate to the connective junctions between a delivery wire assembly and the vaso-occlusive device being implanted using the delivery wire assembly.

### BACKGROUND

Vaso-occlusive devices or implants are used for a wide variety of reasons, including treatment of intra-vascular aneurysms. Commonly used vaso-occlusive devices include soft, helically wound coils formed by winding a platinum (or platinum alloy) wire strand about a “primary” mandrel. The coil is then wrapped around a larger, “secondary” mandrel, and heat treated to impart a secondary shape. For example, U.S. Pat. No. 4,994,069, issued to Ritchart et al., which is fully incorporated herein by reference, describes a vaso-occlusive device that assumes a linear, helical primary shape when stretched for placement through the lumen of a delivery catheter, and a folded, convoluted secondary shape when released from the delivery catheter and deposited in the vasculature.

In order to deliver the vaso-occlusive devices to a desired site in the vasculature, e.g., within an aneurysmal sac, it is well-known to first position a small profile, delivery catheter or “micro-catheter” at the site using a steerable guidewire. Typically, the distal end of the micro-catheter is provided, either by the attending physician or by the manufacturer, with a selected pre-shaped bend, e.g., 45°, 26°, “J”, “S”, or other bending shape, depending on the particular anatomy of the patient, so that it will stay in a desired position for releasing one or more vaso-occlusive device(s) into the aneurysm once the guidewire is withdrawn. A delivery or “pusher” wire is then passed through the micro-catheter, until a vaso-occlusive device coupled to a distal end of the delivery wire assembly is extended out of the distal end opening of the micro-catheter and into the aneurysm. Once in the aneurysm, segments of some vaso-occlusive devices break off to allow more efficient and complete packing. The vaso-occlusive device is then released or “detached” from the end delivery wire assembly, and the delivery wire assembly is withdrawn back through the catheter. Depending on the particular needs of the patient, one or more additional occlusive devices may be pushed through the catheter and released at the same site.

One well-known way to release a vaso-occlusive device from the end of the delivery wire assembly is through the use of an electrolytically severable junction, which is a small exposed section or detachment zone located along a distal end portion of the delivery wire assembly. The detachment zone is typically made of stainless steel and is located just proximal of the vaso-occlusive device. An electrolytically severable junction is susceptible to electrolysis and disintegrates when the delivery wire assembly is electrically charged in the presence of an ionic solution, such as blood or other bodily fluids. Thus, once the detachment zone exits out of the catheter distal end and is exposed in the vessel blood pool of the patient, a current applied through an electrical contact to the conductive pusher wire completes an electrolytic detachment circuit with a return electrode, and the detachment zone disintegrates due to electrolysis.

The vaso-occlusive device is attached to the delivery wire assembly distal of the detachment zone at a main junction. In some vaso-occlusive device delivery systems, a main junction link joins the vaso-occlusive device to the delivery wire assembly and is covered with an adhesive, such as ultraviolet curable glue, wherein the main junction include part of the vaso-occlusive device, part of the delivery wire assembly, the main junction link, and the adhesive.

### SUMMARY

In one embodiment of the disclosed inventions, a vaso-occlusive device delivery system includes a delivery wire assembly having a core wire disposed in a delivery wire lumen, and a vaso-occlusive device having a tapered proximal end and defining a vaso-occlusive device lumen. The vaso-occlusive device further comprises a tether disposed in the lumen, wherein the tether is secured to the core wire. The system may optionally include a link respectively attached to the core wire, the proximal end of the vaso-occlusive device, and the tether. Alternatively or additionally, the vaso-occlusive device is screwed onto the link. The system may also include a clip respectively attached to the core wire and the tether. In some embodiments, the clip is laminated to the core wire. In other embodiments, the distal end of the clip forms a loop to which the tether is attached. In some embodiments, the distal end of the core wire forms a loop to which the tether is attached. In other embodiments, the distal end of the core wire is flattened and restrains the tether on the core wire.

In another embodiment of the disclosed inventions, a vaso-occlusive device delivery system includes a delivery wire assembly, a link attached to a distal end portion of the delivery wire assembly, a transition coil attached to the link and having a transition coil outer diameter, and a vaso-occlusive device attached to the transition coil and having a vaso-occlusive device outer diameter larger than the transition coil outer diameter. In some embodiments, the transition coil is attached to an interior surface of the vaso-occlusive device. In other embodiments, the transition coil is attached to a proximal end surface of the vaso-occlusive device.

In still another embodiment of the disclosed inventions, a vaso-occlusive device delivery system includes a delivery wire assembly having a core wire disposed in a delivery wire lumen, a transition member attached to a distal end portion of the core wire and having a transition member outer diameter, and a vaso-occlusive device defining a lumen having a tether disposed therein and secured to the core wire, the vaso-occlusive device having an outer diameter larger than an out diameter of the transition member. In some embodiments, the transition member is attached to an interior surface of the vaso-occlusive device. In other embodiments, the transition member is attached to a proximal end surface of the vaso-occlusive device. The system may optionally include a clip respectively attached to the core wire and the tether. In some embodiments, the core wire is laminated to the clip. In other embodiments, a distal end of the transition member forms a loop to which the tether is attached. Alternatively or additionally, the transition member comprises an open pitch coil. In other embodiments, the transition member comprises a coil having an open pitch proximal portion and a closed pitch distal portion. In still other embodiments, the transition member comprises a tube having a continuous proximal portion and a slotted distal portion.

Other and further aspects and features of embodiments of the disclosed inventions will become apparent from the ensuing detailed description in view of the accompanying figures.

### BRIEF DESCRIPTION OF THE DRAWINGS

The drawings illustrate the design and utility of embodiments of the disclosed inventions, in which similar elements are referred to by common reference numerals. These drawings are not necessarily drawn to scale.

FIG. 1 illustrates an occlusive coil delivery system, according to one embodiment of the disclosed inventions, with a portion of the core wire shown in phantom for clarity.

FIG. 2 is a longitudinal cross-sectional view of a delivery wire assembly/vaso-occlusive device main junction according to an embodiment of the disclosed inventions.

FIG. 3 illustrates an occlusive coil in a natural state mode, illustrating one exemplary secondary configuration according to an embodiment of the disclosed inventions.

FIGS. 4-7 and 11-14 are detailed longitudinal cross-section views of delivery wire assembly/vaso-occlusive device main junctions constructed according to various embodiments of the disclosed inventions.

FIGS. 8-10 are detailed side views of the core wire/suture junctions constructed according to various embodiments of the disclosed inventions.

### DETAILED DESCRIPTION OF THE ILLUSTRATED EMBODIMENTS

Various embodiments are described hereinafter with reference to the figures. It should be noted that the figures are not drawn to scale and that elements of similar structures or functions are represented by like reference numerals throughout the figures. It should also be noted that the figures are only intended to facilitate the description of the embodiments. They are not intended as an exhaustive description of the invention or as a limitation on the scope of the invention, which is defined only by the appended claims and their equivalents. In addition, an illustrated embodiment needs not have all the aspects or advantages shown. An aspect or an advantage described in conjunction with a particular embodiment is not necessarily limited to that embodiment and can be practiced in any other embodiments even if not so illustrated.

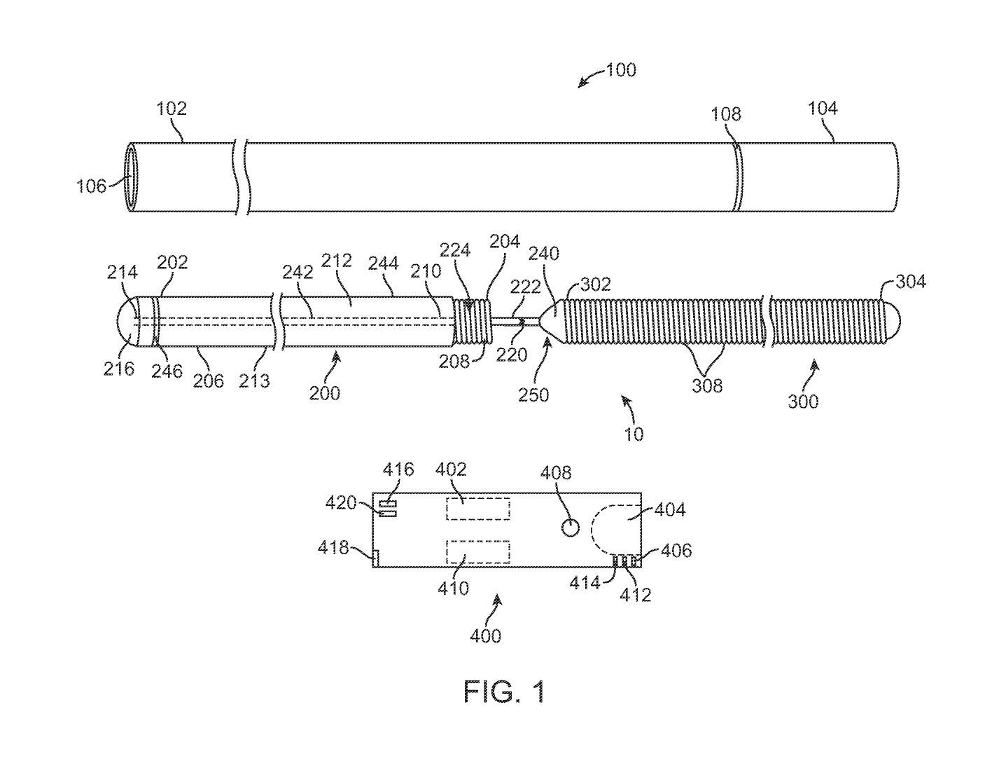

FIG. 1 illustrates a known occlusive coil delivery system 10. The system 10 includes a number of subcomponents or sub-systems. These include a delivery catheter 100, a delivery wire assembly 200, an occlusive coil 300, and a power supply 400. The delivery catheter 100 includes a proximal end 102, a distal end 104, and a lumen 106 extending between the proximal and distal ends 102, 104. The lumen 106 of the delivery catheter 100 is sized to accommodate axial movement of the delivery wire assembly 200. Further, the lumen 106 is sized for the passage of a guidewire (not shown) which may optionally be used to properly guide the delivery catheter 100 to the appropriate delivery site.

The delivery catheter 100 may include a braided-shaft construction of stainless steel flat wire that is encapsulated or surrounded by a polymer coating. By way of non-limiting example, HYDROLENE® is a polymer coating that may be used to cover the exterior portion of the delivery catheter 100. Of course, the system 10 is not limited to a particular construction or type of delivery catheter 100 and other constructions known to those skilled in the art may be used for the delivery catheter 100. The inner lumen 106 may be advantageously coated with a lubricious coating such as PTFE to reduce frictional forces between the delivery catheter 100 and the respective delivery wire assembly 200 and occlusive coil 300 being moved axially within the lumen 106. The delivery catheter 100 may include one or more optional marker bands 108 formed from a radiopaque material that can be used to identify the location of the delivery catheter 100 within the patient's vasculature system using imaging technology (e.g., fluoroscope imaging). The length of the delivery catheter 100 may vary depending on the particular application, but generally is around 150 cm in length. Of course, other lengths of the delivery catheter 100 may be used with the system 10 described herein.

The delivery catheter 100 may include a distal end 104 that is straight as illustrated in FIG. 1. Alternatively, the distal end 104 may be pre-shaped into a specific geometry or orientation. For example, the distal end 104 may be shaped into a “C” shape, an “S” shape, a “J” shape, a 45° bend, a 90° bend. The size of the lumen 106 may vary depending on the size of the respective delivery wire assembly 200 and occlusive coil 300, but generally the OD of the lumen 106 of the delivery catheter 100 (I.D. of delivery catheter 100) is less than about 0.02 inches. The delivery catheter 100 is known to those skilled in the art as a microcatheter. While not illustrated in FIG. 1, the delivery catheter 100 may be utilized with a separate guide catheter (not shown) that aids in guiding the delivery catheter 100 to the appropriate location within the patient's vasculature.

Still referring to FIG. 1, the system 10 includes a delivery wire assembly 200 configured for axial movement within the lumen 106 of the delivery catheter 100. The delivery wire assembly 200 generally includes a proximal end 202 and a distal end 204. The delivery wire assembly 200 includes a delivery wire conduit 224, which has a proximal tubular portion 206 and a distal coil portion 208. The proximal tubular portion 206 may be formed from, for example, a flexible stainless steel hypotube. The distal coil portion 208 may be formed from, for example, stainless steel wire. The distal coil portion 208 may be joined to the proximal tubular portion 206 in an end-to-end arrangement.

Referring to FIG. 1, the core wire 210 is formed from an electrically conductive material such as stainless steel wire. The proximal end 214 of the core wire 210 (shown partially in phantom) is electrically coupled to an electrical contact 216 located at the proximal end 202 of the delivery wire assembly 200. The electrical contact 216 may be formed from a metallic solder (e.g., gold) that is configured to interface with a corresponding electrical contact (not shown) in the power supply 400. The core wire 210 is connected to the delivery wire conduit 224 as described below.

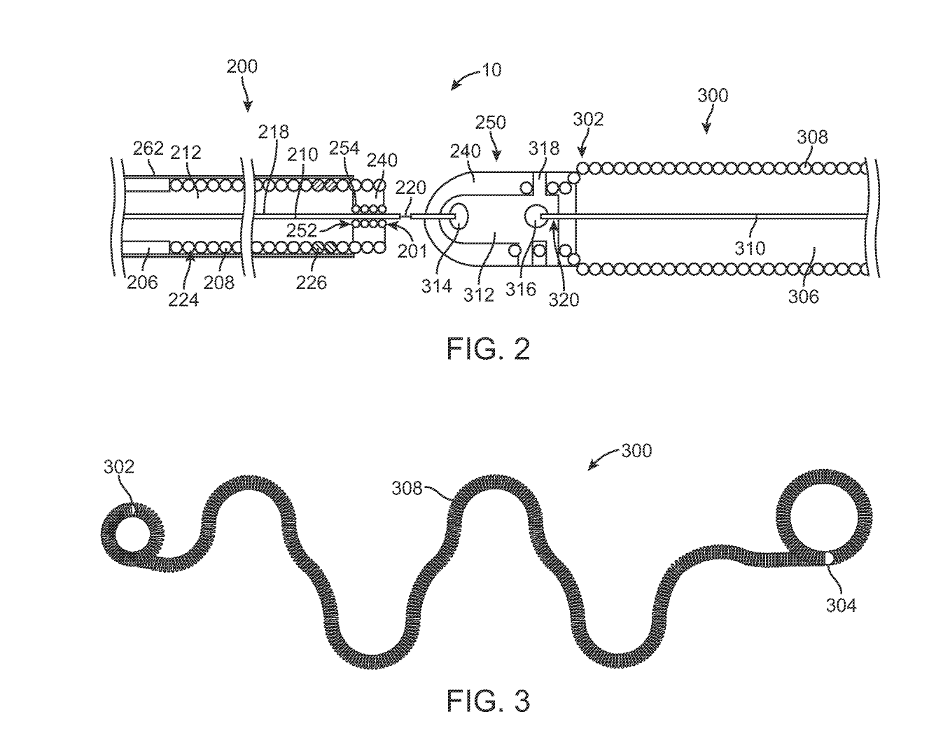

FIG. 2 illustrates a longitudinal cross-sectional view of the delivery wire assembly 200 according to one embodiment. Similar elements of this embodiment are identified with the same reference numbers as discussed above with respect to FIG. 1. The delivery wire assembly 200 includes a proximal end 202 and a distal end 204 and measures between around 184 cm to around 186 cm in length. The delivery wire assembly 200 includes a delivery wire conduit 224 with a proximal tubular portion 206, a distal coil portion 208, and a distal opening 201. The proximal tubular portion 206 may be formed from stainless steel hypotube having an OD of 0.01325 inches and inner diameter (ID) of 0.0075 inches. The length of the hypotube section may be between around 140 cm to around 150 cm, although other lengths may also be used.

As shown in FIG. 2, the delivery wire assembly 200 further includes a core wire 210 that extends from the proximal end 202 of the delivery wire assembly 200 to a location that is distal with respect to the distal end 204 of the delivery wire assembly 200. The core wire 210 is disposed within a conduit lumen 212 that extends within an interior portion of the delivery wire conduit 224. The distal end of the conduit lumen 212 is sealed with a stopper 252. The stopper 252 is made of a stopper coil 254 and an adhesive 240 that secures the stopper coil 254 to the delivery wire conduit 224 and the core wire 210.

A portion of the core wire 210 is advantageously coated with an insulative coating 218. The insulative coating 218 may include polyimide. The entire length of the core wire 210 is coated with an insulative coating 218, except for the proximal end 214 of the core wire 210 that contacts the electrical contact 216, and a small region 220 located in a portion of the core wire 210 that extends distally with respect to the distal end 204 of the delivery wire assembly 200. This latter, “bare” portion of the core wire 210 forms the electrolytic detachment zone 220, which dissolves upon application of electrical current from the power supply 400.

As seen in FIG. 2, a distal coil portion 208 is joined in end-to-end fashion to the distal face of the proximal tubular portion 206. The joining may be accomplished using a weld or other bond. The distal coil portion 208 may have a length of around 39 cm to around 41 cm in length. The distal coil portion 208 may comprise a coil of 0.0025 inches×0.006 inches. The first dimension generally refers to the OD of the coil wire that forms the coil. The latter dimension generally refers to the internal mandrel used to wind the coil wire around to form the plurality of coil winds and is the nominal ID of the coil. One or more marker coils 226 of the distal coil portion 208 may be formed from a radiopaque material. For example, the distal coil portion 208 may include a segment of stainless steel coil (e.g., 3 cm in length), followed by a segment of platinum coil (which is radiopaque and also 3 mm in length), followed by a segment of stainless steel coil (e.g., 37 cm in length), and so on and so forth.

An outer sleeve 262 or jacket surrounds a portion of the proximal tubular portion 206 and a portion of the distal coil portion 208 of the delivery wire conduit 224. The outer sleeve 262 covers the interface or joint formed between the proximal tubular portion 206 and the distal coil portion 208. The outer sleeve 262 may have a length of around 50 cm to around 54 cm. The outer sleeve 262 may be formed from a polyether block amide plastic material (e.g., PEBAX 7233 lamination). The outer sleeve 262 may include a lamination of PEBAX and HYDROLENE® that may be heat laminated to the delivery wire assembly 200. The OD of the outer sleeve 262 may be less than 0.02 inches and advantageously less than 0.015 inches. During manufacturing, the outer sleeve 262 is removed from the very distal end of the delivery wire conduit 224 to form an exposed return cathode.

The core wire 210, which runs through the delivery wire conduit 224, terminates at electrical contact 216 at one end and extends distally with respect to the distal coil portion 208 of the delivery wire conduit 224 to the core wire distal end 222 at the other end. The core wire 210 is coated with an insulative coating 218 such as polyimide except at the electrolytic detachment zone 220 and the proximal segment coupled to the electrical contact 216. The electrolytic detachment zone 220 is located less and half a millimeter (e.g., about 0.02 mm to about 0.2 mm) distally with respect to the distal end of the distal coil portion 208. The core wire 210 may have an OD of around 0.00175 inches.

The occlusive coil 300 includes a proximal end 302, a distal end 304, and a lumen 306 extending there between. The occlusive coil 300 is made from a biocompatible metal such as platinum or a platinum alloy (e.g., platinum-tungsten alloy). A tether 310, such as a suture, extends from the proximal end 302 through the lumen 306 to the distal end 304 where it is connected to the distal end 304 of the occlusive coil 300. The occlusive coil 300 includes a plurality of coil windings 308. The coil windings 308 are generally helical about a central axis disposed along the lumen 306 of the occlusive coil 300. The occlusive coil 300 may have a closed pitch configuration as illustrated in FIGS. 1 and 2.

The occlusive coil 300 generally includes a straight configuration (as illustrated in FIG. 1) when the occlusive coil 300 is loaded within the delivery catheter 100. Upon release, the occlusive coil 300 generally takes a secondary shape which may include three-dimensional helical configurations. FIG. 3 illustrates one exemplary configuration of an occlusive coil 300 in a natural state. In the natural state, the occlusive coil 300 transforms from the straight configuration illustrated in, for instance, FIG. 1 into a secondary shape. The secondary shaped may include both two and three dimensional shapes of a wide variety. FIG. 3 is just one example of a secondary shape of an occlusive coil 300 and other shapes and configurations are contemplated to fall within the scope of the disclosed inventions. Also, the occlusive coil 300 may incorporate synthetic fibers over all or a portion of the occlusive coil 300 as is known in the art. These fibers may be attached directly to coil windings 308 or the fibers may be integrated into the occlusive coil 300 using a weave or braided configuration.

Of course, the system 10 described herein may be used with occlusive coils 300 or other occlusive structures having a variety of configurations, and is not limited to occlusive coils 300 having a certain size or configuration. The distal end 222 of the core wire 210 is connected to the proximal end 302 of the occlusive coil 300 at a main junction 250. It is preferable to apply an adhesive 240 to cover the main junction 250. The adhesive 240 may include an epoxy material which is cured or hardened through the application of heat or UV radiation. For example, the adhesive 240 may include a thermally cured, two-part epoxy such as EPO-TEK® 353ND-4 available from Epoxy Technology, Inc., 14 Fortune Drive, Billerica, Mass. The adhesive 240 encapsulates the main junction 250 and increases its mechanical stability. Additional features and components used to provide mechanical interlock between the delivery wire assembly 200 and occlusive coil 300, while maintaining a smaller OD, are described below in greater detail.

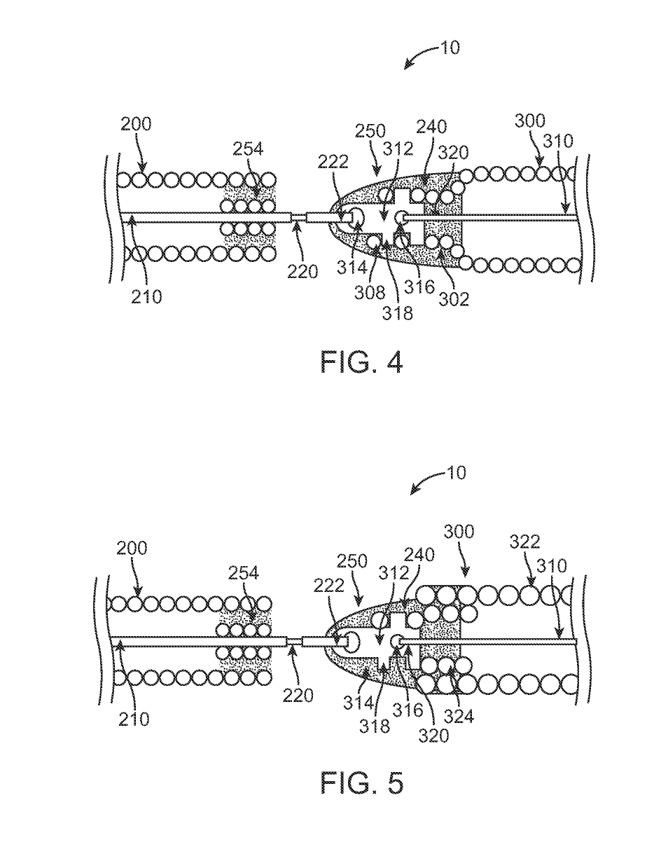

In the embodiment in FIGS. 4-6, the delivery wire assembly 200 and the occlusive coil 300 are connected using a link 312. The link 312 is a small flat body with openings 314, 316 configured to restrain loops of core wire 210 and tether 310 and detents 318 configured to restrain windings of coils. The link 312 can be formed from any biocompatible material. The distal end 222 of the core wire 210 is looped through the proximal opening 314, and the proximal end 320 of the tether 310 is looped through the distal opening 316.

In the embodiment in FIG. 4, the main junction 250 is formed from the distal end 222 of the core wire 210, the link 312, the proximal end 320 of the tether 310, the proximal end 302 of the occlusive coil 300, and the adhesive 240. The proximal end 302 of the occlusive coil 300 is tapered to a smaller OD and screwed onto the link 312. As such, several coil windings 308 at the proximal end 302 of the occlusive coil 300 are interlaced between detents 318 to secure the occlusive coil 300 to the link 312. In embodiments where the OD of the wire forming the occlusive coil 300 is 0.0155 inches (e.g., the −18 coil family) the occlusive coil 300 can taper down to an OD of 0.012 inches at the proximal end 302. Accordingly, the OD of the main junction 250 can be from 0.014 to 0.015 inches.

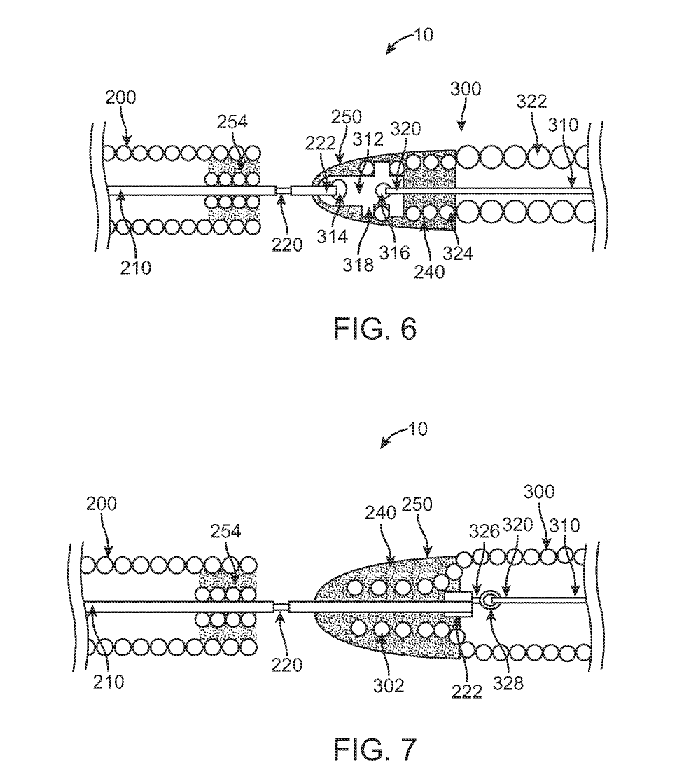

In the embodiments in FIGS. 5 and 6, the occlusive coil 300 includes a larger OD main coil 322 and a smaller OD transition coil 324. The transition coil 324 is wound from wire having the same composition as the wire used to form the main coil 322. However, the wire forming the transition coil 324 has a smaller OD that the wire used to form the main coil 322.

In the embodiment in FIG. 5, the main junction 250 is formed from the distal end 222 of the core wire 210, the link 312, a transition coil 324, the proximal end 320 of the tether 310, the proximal end of the main coil 322, and adhesive 240. The distal end of the transition coil 324 is disposed in the proximal end of the main coil 322. The outside surface of the transition coil 324 is attached to the interior surface of the main coil 322 by techniques such as laser melting, and laser tack, spot and continuous welding.

In the embodiment in FIG. 6, the main junction 250 is formed from the distal end 222 of the core wire 210, the link 312, a transition coil 324, the proximal end 320 of the tether 310, and adhesive 240. The distal end surface of the transition coil 324 is attached to the proximal end surface of the main coil 322 by butt welding using the techniques described above.

In the embodiment in FIG. 7, the main junction 250 is formed from the distal end 222 of the core wire 210, a clip 326 having a loop 328, the proximal end 320 of the tether 310, the proximal end 302 of the occlusive coil 300, and adhesive 240. The distal end 222 of the core wire 210 is laminated to the clip 326. The proximal end 320 of the tether 310 is threaded through the loop 328 in the clip 326. Further, the proximal end 302 of the occlusive coil 300 is tapered to a smaller OD. Moreover, the proximal end 302 of the occlusive coil 300 has an open pitch, which allows the adhesive 240 to penetrate into the occlusive coil 300 and secure the distal end 222 of the core wire 210 to the occlusive coil 300.

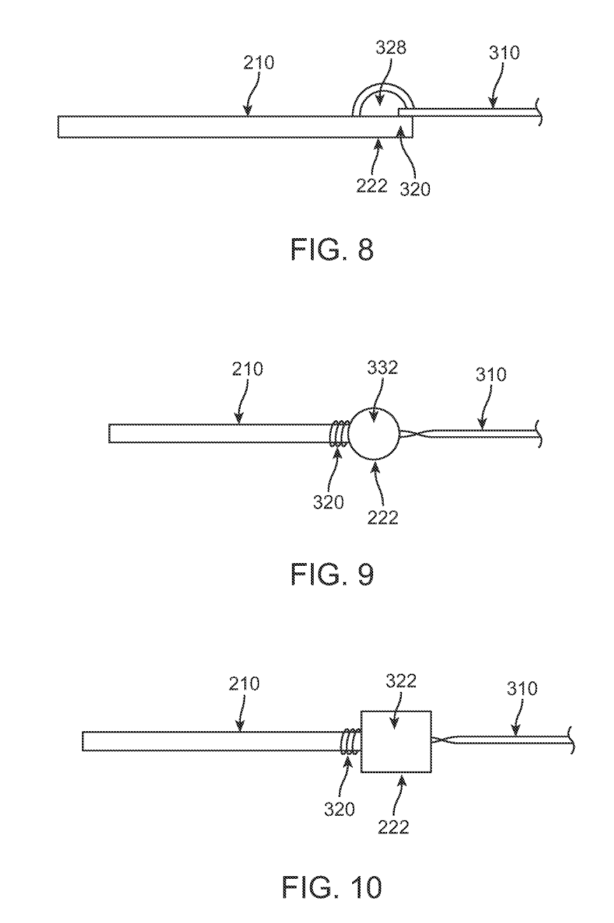

FIGS. 8-10 depict alternative mechanisms for securing the tether 310 to the core wire 210 for use with the embodiment in FIG. 7. In FIG. 8, the distal end 222 of the core wire 210 is bent back on itself to form a loop 328 through which the proximal end 320 of the tether 310 is threaded. In FIGS. 9 and 10, the proximal end 320 of the tether 310 is looped around the distal end 222 of the core wire 210. Further, the distal end 222 of the core wire 210 is flattened to form a flange 332, which restrains the tether 310 against distal movement.

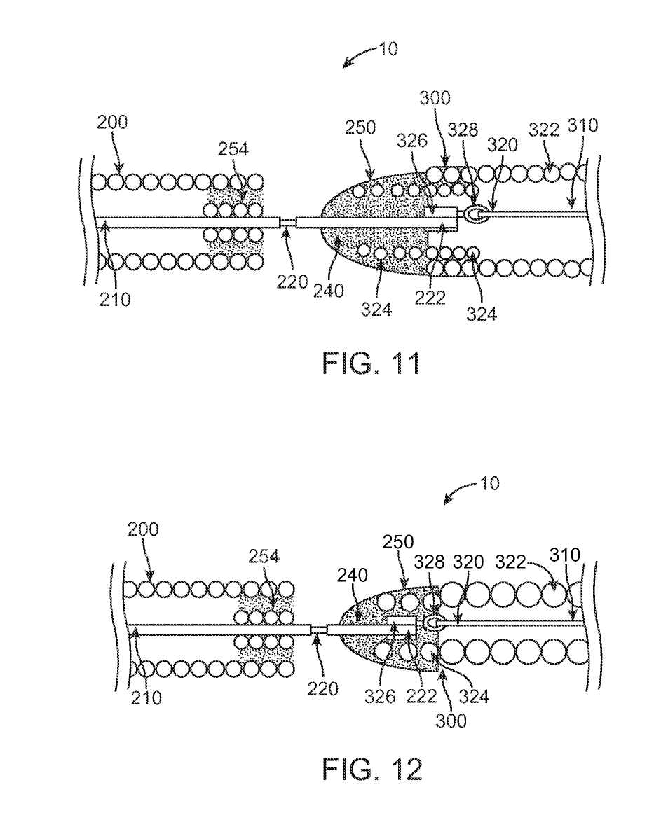

In the embodiments in FIGS. 11 to 13, the occlusive coil 300 includes a larger OD main coil 322 and a smaller OD transition coil 324. In the embodiment in FIG. 11, the main junction 250 is formed from the distal end 222 of the core wire 210, the transition coil 324, a clip 326 having a loop 328, the proximal end 320 of the tether 310, and adhesive 240. The distal end 222 of the core wire 210 is laminated to the clip 326. The proximal end 320 of the tether 310 is threaded through the loop 328 in the clip 326. Further, the distal end of the transition coil 324 is disposed in the proximal end of the main coil 322. The outside surface of the transition coil 324 is attached to the interior surface of the main coil 322 by techniques such as laser welding. Moreover, the proximal end of the transition coil 324 has an open pitch, which allows the adhesive 240 to penetrate into the occlusive coil 300 and secure the distal end 222 of the core wire 210 to the transition coil 324. FIG. 12 depicts an embodiment similar to that in FIG. 11 except that the distal end surface of the transition coil 324 is attached to the proximal end surface of the main coil 322 by butt welding using the techniques described above.

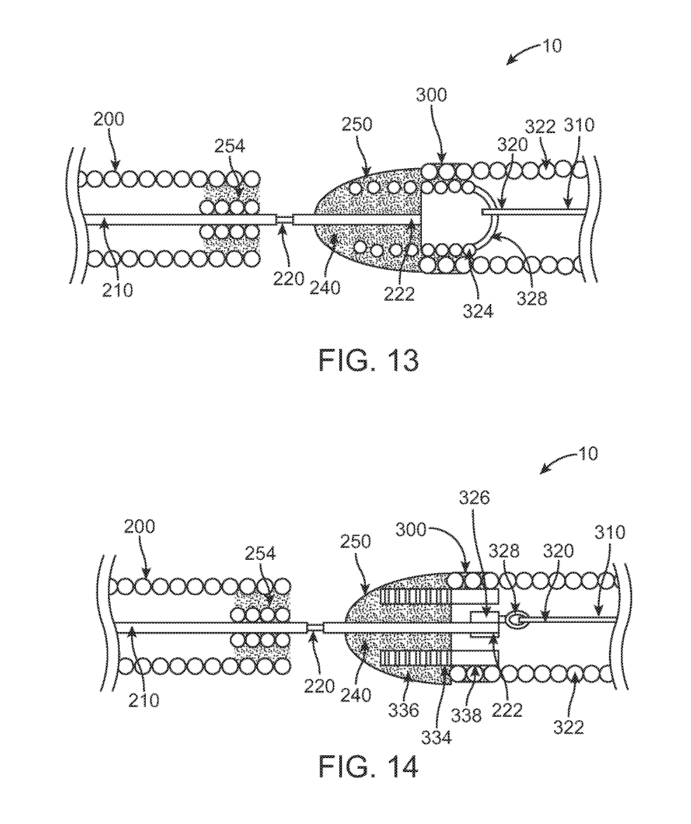

In the embodiment in FIG. 13, the main junction 250 is formed from the distal end 222 of the core wire 210, the transition coil 324, the proximal end 320 of the tether 310, and adhesive 240. The distal end of the transition coil 324 is disposed in the proximal end of the main coil 322. The outside surface of the transition coil 324 is attached to the interior surface of the main coil 322 by techniques such as laser welding. The proximal end of the transition coil 324 has an open pitch, allowing the adhesive 240 to penetrate into the occlusive coil 300 and secure the distal end 222 of the core wire 210 to reach the transition coil 324. The distal end of the transition coil 324 is stretched to form a loop 328 through which the proximal end 320 of the tether 310 is threaded.

In the embodiment in FIG. 14, the occlusive coil 300 includes a larger OD main coil 322 and a smaller OD transition tube 334. The main junction 250 is formed from the distal end 222 of the core wire 210, the transition tube 334, a clip 326 having a loop 328, the proximal end 320 of the tether 310, and adhesive 240. The distal end 222 of the core wire 210 is laminated to the clip 326. The proximal end 320 of the tether 310 is threaded through the loop 328 in the clip 326. Further, the distal end of the transition tube 334 is disposed in the proximal end of the main coil 322. The outside surface of the transition tube 334 is attached to the interior surface of the main coil 322 by techniques such as laser welding. Moreover, the transition tube 334 has a slotted proximal end 336 and a continuous distal end 338. The slotted proximal end 336 allows the adhesive 240 to penetrate into the occlusive coil 300 and secure the distal end 222 of the core wire 210 to the transition coil 324.

As shown in FIGS. 4 to 14, the core wire 210 is attached to the tether 310 using various mechanisms. Accordingly, prior to the occlusive coil's complete exit from the distal end 104 of the catheter 100, the occlusive coil 300 can be withdrawn proximally into the catheter 100 by pulling the delivery wire assembly 200, which in turn pulls on the core wire 210, the tether 310, and the occlusive coil 300. When the delivery wire assembly 200 and the occlusive coil 300 are pushed distally into the delivery catheter 100, the distal end 222 of the core wire 210 carries the load between the two parts. Other mechanisms may be used to carry the load when the delivery wire assembly 200 and the occlusive coil 300 are pushed distally.

It should be appreciated that the materials for forming the occlusive coil 300 are not be limited to the examples described previously. In any of the embodiments described herein, the material for the coil 300 may be a radio-opaque material such as a metal or a polymer. Also, in other embodiments, the material for the coil 300 may be rhodium, palladium, rhenium, as well as tungsten, gold, silver, tantalum, and alloys of these metals. These metals have significant radio-opacity and in their alloys may be tailored to accomplish an appropriate blend of flexibility and stiffness. They are also largely biologically inert. Also, any materials which maintain their shape despite being subjected to high stress may be used to construct the coil 300.

For example, certain “super-elastic alloys” include various nickel/titanium alloys (48-58 atomic % nickel and optionally containing modest amounts of iron); copper/zinc alloys (38-42 weight % zinc); copper/zinc alloys containing 1-10 weight % of beryllium, silicon, tin, aluminum, or gallium; or nickel/aluminum alloys (36-38 atomic % aluminum), may be used. In further embodiments, titanium-nickel alloy known as “nitinol” may be used to form the coil 300. These are very sturdy alloys which will tolerate significant flexing without deformation even when used as very small diameter wire.

In any of the embodiments described herein, the wire used to form the coil 300 may have a cross-sectional dimension that is in the range of 0.00002 and 0.01 inches. The coil 300 may have a cross-sectional dimension between 0.003 and 0.03 inches. In various embodiments, the wires can have any geometry, such as square, rectangle, or circle. For neurovascular applications, the diameter of the coil may be anywhere from 0.008 to 0.018 inches. In other embodiments, the wires may have other cross-sectional dimensions, and the coil 300 may have other cross-sectional dimensions. In some embodiments, the wire for forming the coil 300 should have a sufficient diameter to provide a hoop strength to the resulting occlusive coil 300 sufficient to hold the coil 300 in place within the chosen body site, lumen or cavity, without substantially distending the wall of the site and without moving from the site as a result of the repetitive fluid pulsing found in the vascular system.

In any of the embodiments described herein, the axial length of the coil 300 may be in the range of 0.5 to 100 cm, and more preferably, in the range of 2.0 to 40 cm. Depending upon use, the coil 300 may have 10-75 turns per centimeter, or more preferably 10-40 turns per centimeter. In other embodiments, the coil 300 may have other lengths and/or other number of turns per centimeter.

As shown in FIG. 1, the system 10 further includes a power supply 400 for supplying direct current to the core wire 210, which contains the electrolytic detachment zone 220. In the presence of an electrically conductive fluid (including a physiological fluid such as blood, or an electrically conductive flushing solution such as saline), activation of the power supply 400 causes electrical current to flow in a circuit including the core wire electrical contact 216, the core wire 210, the electrolytic detachment zone 220, and a return electrode (not shown). After several seconds (generally less than about 10 seconds), the sacrificial electrolytic detachment zone 220 dissolves, and the occlusive coil 300 separates form the core wire 210.

The power supply 400 preferably includes an onboard energy source, such as batteries (e.g., a pair of AAA batteries), along with drive circuitry 402. The drive circuitry 402 may include one or more microcontrollers or processors configured to output a driving current. The power supply 400 illustrated in FIG. 1 includes a receptacle 404 configured to receive and mate with the proximal end 202 of the delivery wire assembly 200. Upon insertion of the proximal end 202 into the receptacle 404, the electrical contact 216 disposed on the delivery wire assembly 200 electrically couple with corresponding contacts (not shown) located in the power supply 400.

A visual indicator 406 (e.g., LED light) is used to indicate when the proximal end 202 of delivery wire assembly 200 has been properly inserted into the power supply 400. Another visual indicator 420 is activated if the onboard energy source needs to be recharged or replaced. The power supply 400 includes an activation trigger or button 408 that is depressed by the user to apply the electrical current to the sacrificial electrolytic detachment zone 220. Once the activation trigger 408 has been activated, the driver circuitry 402 automatically supplies current until detachment occurs. The drive circuitry 402 typically operates by applying a substantially constant current, e.g., around 1.5 mA.

The power supply 400 may include optional detection circuitry 410 that is configured to detect when the occlusive coil 300 has detached from the core wire 210. The detection circuitry 410 may identify detachment based upon a measured impedance value. A visual indicator 412 may indicate when the power supply 400 is supplying adequate current to the sacrificial electrolytic detachment zone 220. Another visual indicator 414 may indicate when the occlusive coil 300 has detached from the core wire 210. As an alternative to the visual indicator 414, an audible signal (e.g., beep) or even tactile signal (e.g., vibration or buzzer) may be triggered upon detachment. The detection circuitry 410 may be configured to disable the drive circuitry 402 upon sensing detachment of the occlusive coil 300.

The power supply 400 may contain another visual indicator 416 that indicates to the operator when non-bipolar delivery wire assembly 200 is inserted into the power supply 400. Non-bipolar delivery wire assemblies 200 use a separate return electrode that typically is in the form of a needle that was inserted into the groin area of the patient. The power supply 400 is configured to detect when a non-bipolar delivery wire assembly 200 has been inserted, which causes the visual indicator 416 (e.g., LED) is turned on and the user is advised to insert the separate return electrode (not shown in FIG. 1) into a port 418 located on the power supply 400.

What is claimed is:

1. A vaso-occlusive device delivery system, comprising:
a delivery wire assembly having a core wire disposed in a delivery wire lumen; and
a vaso-occlusive device having a tapered proximal end and defining a vaso-occlusive device lumen, the vaso-occlusive device further comprising a tether disposed in the lumen, wherein the tether is secured to the core wire.

2. The vaso-occlusive device delivery system of claim 1, further comprising a link respectively attached to the core wire, the proximal end of the vaso-occlusive device, and the tether.

3. The vaso-occlusive device delivery system of claim 2, wherein the vaso-occlusive device is screwed onto the link.

4. The vaso-occlusive device delivery system of claim 1, further comprising a clip respectively attached to the core wire and the tether.

5. The vaso-occlusive device delivery system of claim 4, wherein the clip is laminated to the core wire.

6. The vaso-occlusive device delivery system of claim 5, wherein a distal end of the clip forms a loop to which the tether is attached.

7. The vaso-occlusive device delivery system of claim 4, wherein a distal end of the core wire forms a loop to which the tether is attached.

8. The vaso-occlusive device delivery system of claim 4, wherein the distal end of the core wire is flattened and restrains the tether on the core wire.

9. A vaso-occlusive device delivery system, comprising:
a delivery wire including a core wire;
a link attached to a distal end portion of the core wire;
a transition coil attached to the link and having a transition coil outer diameter; and
a vaso-occlusive device attached to the transition coil and having a vaso-occlusive device outer diameter larger than the transition coil outer diameter.

10. The vaso-occlusive device delivery system of claim 9, wherein the transition coil is attached to an interior surface of the vaso-occlusive device.

11. The vaso-occlusive device delivery system of claim 9, wherein the transition coil is attached to a proximal end surface of the vaso-occlusive device.

12. A vaso-occlusive device delivery system, comprising:
a delivery wire assembly having a core wire disposed in a delivery wire lumen;
a transition member attached to a distal end portion of the core wire and having a transition member outer diameter; and
a vaso-occlusive device defining a lumen having a tether disposed therein and secured to the core wire, the vaso-occlusive device having an outer diameter larger than an outer diameter of the transition member.

13. The vaso-occlusive device delivery system of claim 12, wherein the transition member is attached to an interior surface of the vaso-occlusive device.

14. The vaso-occlusive device delivery system of claim 12, wherein the transition member is attached to a proximal end surface of the vaso-occlusive device.

15. The vaso-occlusive device delivery system of claim 12, further comprising a clip respectively attached to the core wire and the tether.

16. The vaso-occlusive device delivery system of claim 15, wherein the core wire is laminated to the clip.

17. The vaso-occlusive device delivery system of claim 12, wherein a distal end of the transition member forms a loop to which the tether is attached.

18. The vaso-occlusive device delivery system of claim 12, wherein the transition member comprises an open pitch coil.

19. The vaso-occlusive device delivery system of claim 12, wherein the transition member comprises a coil having an open pitch proximal portion and a closed pitch distal portion.

20. The vaso-occlusive device delivery system of claim 12, wherein the transition member comprises a tube having a continuous proximal portion and a slotted distal portion.
